SEQUENCE LISTING (4 sequences)

SEQ ID NO: 1 - DNA, 1015 bp, Vitis vinifera
  misc_feature 1..1015: note = clone4-RNPdmr6-2
  source 1..1015: mol_type = unassigned DNA
atggattctaaggtcctttccaccggaatcccatttaccactttgccggagaactacatcagaccagaatccgagaggcctagactctccgaaattgccgactgcgaaaatgttcctatcatcgacctgagttgtgacgatcgagcccaaattattgagcaactcgctgatgcttgtagccgttatggcttctttcaggttattaatcatggggtgtctgcggaagccatcgagaaaatgctacatgtggctaatgagtttttccagttacctgtggaagagaagatgaagttgtattcagatgacccatcaaagacaatgaggttgtctaccagtttcaatgtcaaaaaggagaaagtgcacaattggagagattatcttagacttcattgccatcctttggagcaatatatgccagagttggccctccaatcctcctgagttcaaggataccgtgagcaattactgtgtagaggttcgacaacttgggcacagactggaagaagccataggagagagtttaggcttggaaaaagattatataagaaatacattgggtgaacaagggcaacatatggcggtaaactattatccaccttgtcccgagccagagttaacttacggactgccagcacatacggaccccaatgctcttacaattcttcttcaagactcacatgtcgcgggtcttcaagtccttaaggacggcaagtgggttgctgttaaaccccacccaggtgcttttgttgttaacattggcgatcaattgcaggcattgagtaatggcaagtacagaagtgtatggcaccgagccactgttaatgtcggtaaggcaaggatgtcgattgcttctttcctctgcccaagcgatgatgcgctcattagtccagcaagggccctcacggatgaggggtctgcagccatatacaggagtttcacgtatgcagagtattacaaaaagttctggagcaggaaccttgaccaagagcactgtttggaagtttttaagaactag

SEQ ID NO: 2 - DNA, 1013 bp, Vitis vinifera
  misc_feature 1..1013: note = clone3-RNPdmr6-2
  source 1..1013: mol_type = unassigned DNA
atggattctaaggtcctttccaccggaatcccatttaccactttgccggagaactacatcagaccagaatccgagaggcctagactctccgaaattgccgactgcgaaaatgttcctatcatcgacctgagttgtgacgatcgagcccaaattattgagcaactcgctgatgcttgtagccgttatggcttctttcaggttattaatcatggggtgtctgcggaagccatcgagaaaatgctacatgtggctaatgagtttttccagttacctgtggaagagaagatgaagttgtattcagatgacccatcaaagacaatgaggttgtctaccagtttcaatgtcaaaaaggagaaagtgcacaattggagagattatcttagacttcattgccatcctttggagcaatatatgccagagggccctccaatcctcctgagttcaaggataccgtgagcaattactgtgtagaggttcgacaacttgggcacagactggaagaagccataggagagagtttaggcttggaaaaagattatataagaaatacattgggtgaacaagggcaacatatggcggtaaactattatccaccttgtcccgagccagagttaacttacggactgccagcacatacggaccccaatgctcttacaattcttcttcaagactcacatgtcgcgggtcttcaagtccttaaggacggcaagtgggttgctgttaaaccccacccaggtgcttttgttgttaacattggcgatcaattgcaggcattgagtaatggcaagtacagaagtgtatggcaccgagccactgttaatgtcggtaaggcaaggatgtcgattgcttctttcctctgcccaagcgatgatgcgctcattagtccagcaagggccctcacggatgaggggtctgcagccatatacaggagtttcacgtatgcagagtattacaaaaagttctggagcaggaaccttgaccaagagcactgtttggaagtttttaagaactag

SEQ ID NO: 3 - DNA, 1012 bp, Vitis vinifera
  misc_feature 1..1012: note = clone2-RNPdmr6-2
  source 1..1012: mol_type = unassigned DNA
atggattctaaggtcctttccaccggaatcccatttaccactttgccggagaactacatcagaccagaatccgagaggcctagactctccgaaattgccgactgcgaaaatgttcctatcatcgacctgagttgtgacgatcgagcccaaattattgagcaactcgctgatgcttgtagccgttatggcttctttcaggttattaatcatggggtgtctgcggaagccatcgagaaaatgctacatgtggctaatgagtttttccagttacctgtggaagagaagatgaagttgtattcagatgacccatcaaagacaatgaggttgtctaccagtttcaatgtcaaaaaggagaaagtgcacaattggagagattatcttagacttcattgccatcctttggagcaatatatgccagaggccctccaatcctcctgagttcaaggataccgtgagcaattactgtgtagaggttcgacaacttgggcacagactggaagaagccataggagagagtttaggcttggaaaaagattatataagaaatacattgggtgaacaagggcaacatatggcggtaaactattatccaccttgtcccgagccagagttaacttacggactgccagcacatacggaccccaatgctcttacaattcttcttcaagactcacatgtcgcgggtcttcaagtccttaaggacggcaagtgggttgctgttaaaccccacccaggtgcttttgttgttaacattggcgatcaattgcaggcattgagtaatggcaagtacagaagtgtatggcaccgagccactgttaatgtcggtaaggcaaggatgtcgattgcttctttcctctgcccaagcgatgatgcgctcattagtccagcaagggccctcacggatgaggggtctgcagccatatacaggagtttcacgtatgcagagtattacaaaaagttctggagcaggaaccttgaccaagagcactgtttggaagtttttaagaactag

SEQ ID NO: 4 - DNA, 1013 bp, Solanum tuberosum
  misc_feature 1..1013: note = clone1-RNPdmr6-1
  source 1..1013: mol_type = unassigned DNA
atggaaacgaaagttatttccagcggaatccaccactctactctccctcaaagttacatccgaccgaatccgataggccacgtctatcggatgtggtcgattgcgaaaatgttccaataattgacttaggttgcggagaccaagctcaaataatccgtctaattggagaagcttgtcaaacttatggtttctttcaggtaattaatcatggtgtaccaaaggaagttgtagagaaaatgctaggggtagctggggaatttttcaatctaccagtagaagagaagctaaaattgtattcagatgatccttcaaagaccatgagattatctactagttttaatgttaaaaaggagacagttcataattggagagattatctcagacttcattgtcatcctctggagaaatatgctcctgaatggccttctaatccatcgtctttcagggatatcgtgagcagatattgcacggaagttcgacaactcggatttagattggaggaagccatagcagagagcctgggcttagagaaagagtgtattaaagatgtattgggagaacaaggccaacatatggctatcaatttttatcctccttgtccacaaccagaactcacatatgggcttccggcccatactgatccaaattcacttacaattcttcttcaagacttgcaagtttctggtcttcaagttcttaaagatggtaaatggttggctgtcaaacctcaaccagatgcctttgtcattaatcttggtgatcaattgcaggcagtaagtaacggtaagtacaaaagtgtatggcatcgagctattgtgaattcagatcaagctaggatgtcagtggcttcgttcctatgtccgtgcgatagcgcgaaaatcagtgctccaaaactcctgacagaagatggatctccagtcatttatcaggacttcacgtatgctgagtattacaagaagttctggagcaggaatttggaccaggaacattgtttggaacttttcaagaattaa

(19) United States
(12) Patent Application Publication
(10) Pub. No.: US 2024/0318191 A1
(43) Pub. Date: Sep. 26, 2024

(54) Method for the Generation of Genome Edited Protoplasts from Clonally Propagated Plant Tissue

(71) Applicants: SciENZA Biotechnologies 4 B.V., Enkhuizen (NL); Fondazione Edmund Mach, San Michele all'Adige (IT)

(72) Inventors: Simone Scintilla, San Michele all'Adige (IT); Umberto Salvagnin, San Michele all'Adige (IT); Lisa Giacomelli, San Michele all'Adige (IT); Katja Gucek Cabral, Enkhuizen (NL); Tieme Zeilmaker, Enkhuizen (NL)

(21) Appl. No.: 18/578,868

(22) PCT Filed: Jul. 12, 2022
(86) PCT No.: PCT/EP2022/069502

§ 371 Date: Jan. 12, 2024

(30) Foreign Application Priority Data
Jul. 14, 2021 (NL) 2028724

(51) Int. Cl. C12N 15/82; C12N 9/22; C12N 15/11

(52) U.S. Cl. CPC C12N 15/8213; C12N 9/22; C12N 15/111; C12N 2310/20

(57) ABSTRACT

Provided herein is a method for the generation of genome edited protoplasts from clonally propagated plant tissue for generation of whole genome edited clonally propagated plants. Also provided herein are genome edited plants obtainable by said method, more specifically genome edited clonally propagated plants, preferably potato plants or woody plants such as grapevine plants.

### CROSS-REFERENCE TO RELATED APPLICATIONS

This application is the United States national phase of International Application No. PCT/EP2022/069502 filed Jul. 12, 2022, and claims priority to The Netherlands Patent Application No. 2028724 filed Jul. 14, 2021, the disclosures of which are hereby incorporated by reference in their entireties.

### SEQUENCE LISTING

The Sequence Listing associated with this application is filed in electronic format via Patent Center and is hereby incorporated by reference into the specification in its entirety. The name of the file containing the Sequence Listing is 2309346.xml. The size of the file is 9,943 bytes, and the file was created on Dec. 14, 2023.

### BACKGROUND OF THE INVENTION

### Field of the Invention

The present invention relates to a method for the generation of genome edited protoplasts from clonally propagated plant tissue for generation of whole genome edited clonally propagated plants. The present invention further relates to genome edited plants obtainable by said method, more specifically genome edited clonally propagated plants, preferably potato plants or woody plants such as grapevine plants.

### Description of Related Art

Genome editing technology allows for precise alterations in plant genomes, facilitating targeted changes of the genome of crop plants. For example, CRISPR-Cas is a very promising gene editing technology that allows very precise changes in the genome of a plant and this genome editing method has widely extended the field of application in the field of plant breeding. This system is based on a relatively simple technology which allows recognizing the DNA-editing site through a complementary RNA sequence, and makes possible the insertion, deletion or even just the modification of one nucleotide. CRISPR-Cas can be introduced inside a cell either in the form of DNA coding for the entire CRISPR system or directly, as ribonucleoprotein (RNP). In particular, direct cellular delivery of RNPs opens attractive scenarios as it would potentially lead to a specific and minimal mutation with no trace of exogenous DNA. With this perspective, the interest on the application of a CRISPR-Cas technology in plants, potentially of increased acceptance by consumers with respect to classic GMOs, is constantly increasing.

Several efficient methods for delivering the CRISPR-Cas machinery into the animal cell are known that enable gene editing inside the cell, however since plant cells (unlike mammalian cells) comprise a cell wall preventing introduction of DNA into the cell, such delivery methods are less efficient and less successful in plants. This makes the utilization of gene editing approaches, including CRISPR-Cas more difficult for plants. For gene editing of plant cells, particle bombardment can be used where nanoparticle bullets loaded with biological material are “shot” into plant tissues to overpass the cell wall barrier and release the nanoparticles-loaded biological cargo to induce genome editing. However, various physical parameters severely affect the efficiency of this strategy. In particular, as not all the cells would be hit by bullets, the downstream regeneration process might give rise to plants in which some tissue portions may exhibit genetic heterogeneity (i.e. chimerism). Furthermore, in seed-propagated crops such as lettuce, cucumber, tomato, etc., the gene editing machinery, such as CRISPR-Cas, or the DNA sequences encoding for it are mainly introduced into the plant cell using Agrobacterium tumefaciens, wherein after gene editing the editing machinery is eliminated from the plants and the modified plants can subsequently be selected. However, this strategy employs exogenous plasmid DNA, containing portions of DNA derived from Agrobacterium, which remain integrated in the cellular plant DNA upon transformation.

Furthermore, for clonally propagated plants wherein the cultivated crop is obtained by asexual reproduction, it is not possible to eliminate the CRISPR-Cas sequences by crossing and segregation, while maintaining the plant's performance and genetic make-up. Examples of commercially relevant clonally propagated plants are apple, grape, tart cherry, yam, cassava, sweet potato, taro, and potato (Solanum tuberosum) as well as most plants belonging to the genus Prunus and woody plants grown for fruit production. For example with grapevines, only a limited number of grapevine cultivars are maintained by vegetative propagation to preserve the intrinsic quality and have been used for many decades to produce high quality wine. The ability to introduce new traits, such as disease resistance, using this present precise genome editing technology into these grapevine cultivars without altering their essential characters and preserve the intrinsic quality, is essential.

Protoplast culture seems to provide a promising method for producing non-chimeric gene edited plants of clonally propagated species. A protoplasts is a modified plant cell, wherein their cell wall has been removed providing a single “naked” plant cell. Since the protoplast cells are missing their protective cell wall, these cells are more suitable for gene editing techniques that are also used for mammalian cells, for example by means of classic methodologies such as PEG infiltration, electroporation or lipofection. However, because protoplasts do not comprise the protective cell wall, these cells are very delicate and require careful handling and regulation of the culturing conditions, such as the culture media in which they are cultivated. For example, if the osmotic pressure of the culture medium is not adjusted to match the osmotic pressure within the protoplast, it will implode or burst.

Although protoplasts can be easily obtained from several tissues, its application to plants and especially woody plants (e.g. grapevine, apple) is hampered by low editing efficiencies and unsuccessful regenerative process, which usually stops after few cellular divisions. Successful isolation of protoplasts from clonally propagated plants, such as grape has been demonstrated, however production of mini-calli from these protoplasts has proven to be inefficient, with less than 5% of the isolated protoplasts forming calli. Furthermore, at present no method is available to generate whole clonally propagated plant species, such as grapevine plants, from genome edited cells, such as genetically edited embryogenic callus protoplasts.

Considering the above, there is a need in the art for a method for the generation of genome edited clonally propagated whole plants, for example grapevine plants, that ensures genetic homogeneity in the obtained plants. In addition there is a need in the art for protoplast-derived whole plants that have been genetically edited without altering their essential characters and preserve the intrinsic quality of the plant.

### SUMMARY OF THE INVENTION

It is an object of the present invention, amongst other objects, to address the above need in the art. The object of present invention, amongst other objects, is met by the present invention as outlined in the appended claims.

Specifically, the above object, amongst other objects, is met, according to a first aspect, by the present invention by a method for the generation of genome edited protoplasts from clonally propagated plant tissue for generation of whole plants, wherein the method comprises the steps of;
a) providing a cell suspension from clonally propagated plant tissue selected from the group consisting of embryogenic callus, non-embryogenic callus, and/or leaf cells;
b) contacting the cell suspension with an enzyme composition for digestion of the plant cell wall;
c) isolating protoplast from the cell suspension and washing of the isolated protoplast with a wash solution;
d) genome editing of the isolated protoplast by delivery of biological material into the protoplast;
e) generation of mini-callus colonies from the genome edited protoplast.
   Protoplast culture provides one of the best methods for producing non-chimeric gene edited plants for clonally propagated species such as grape (Vitis vinifera) or potato (Solanum tuberosum). The method generates protoplasts from embryogenic, non-embryogenic or leaf tissue and stimulates the protoplast to reform a cell wall, and to divide and develop into cell colonies, and to form into embryos and eventually to germinate into whole plants. The method of present invention provides for the generation of genetically modified or edited whole plants.

The invention is a DNA-free methodology by which single cells can be transfected with CRISPR-Cas components for genome editing purposes and, upon cellular division and differentiation, provide completely edited plants. The method of present invention allows the isolation of protoplasts of clonally propagated plants, such as grapevine, formation of mini-calli, and subsequent regeneration of whole protoplast-derived plants that have been genetically modified or edited. Using the method of present invention for the generation of genome edited protoplasts from clonally propagated plant tissue for generation of whole plants, for example a grapevine plant, which in general has not been genetically modified in decades, can be gene edited to improve specific traits without altering the genetic integrity of the clone used for editing purposes. For example, specific genes can be edited to achieve improved disease resistance of grape vine clones which prevent excessive spraying of vineyard in the future. The method provides a non-Agrobacterium-mediated, non-integrating gene editing method for further development of non-chimeric gene edited clonally propagated crops, such as grapevine, apple and potatoes. Furthermore, the method may also be used in potato, chicory, lettuce, tobacco and tomato. The single cell methodology is suitable for DNA-free gene editing, potentially leading to a specific and minimal mutation with no trace of exogenous DNA. With respect to classic GMOs, these products will potentially find greater consumers acceptance.

According to another preferred embodiment, the present invention relates to the method, wherein the clonally propagated plant tissue is selected from the group consisting of, grapevine (Vitis vinifera), apple, cherry, yam, cassava, sweet potato, taro, potato (Solanum tuberosum), and woody plant tissue, preferably grapevine or potato tissue.

According to a preferred embodiment, the present invention relates to the method, wherein the enzyme composition is comprised of 0.5 to 3% (w/v), preferably 0.75 to 2%, more preferably 1 to 1.5% cellulase, 0.1 to 0.6% (w/v) hemicellulase, preferably 0.2 to 0.5%, more preferably 0.3 to 0.45%, and 0.1 to 0.5% (w/v) macerozyme R-10, preferably 0.25 to 0.4%, more preferably 0.3 to 0.35%. The method of present invention comprises the use of a lytic enzymes mixture comprising various enzymes for improved plant cell wall digestion, breakdown or disintegration, wherein the lytic enzyme mixture is optimized to reflect the different carbohydrate polymers of the cell wall (for example 1% (w/v) cellulase, 0.3% (w/v) hemicellulase, and 0.3% (w/v) Macerozyme R-10 for grapevine derived tissue). Hemicellulase, a component of the enzymatic mixture is included as a further contribution to the enzymatic mixture. Compositions in cellulase, hemicellulase and macerozyme were tested to find the most optimal ratio for affecting plant tissue, since for example grapevine cell wall composition is variable depending on the cell specialization. When using mixtures outside the claimed range, the method of present invention did not result in optimal protoplasts that could be genetically edited and used for the generation of gene edited whole plants.

According to another preferred embodiment, the present invention relates to the method, wherein the wash solution is osmotically adjusted to substantially correspond to the osmotic values of the isolated protoplast, and wherein the concentration of mannitol is at most 0.5 M, preferably at most 0.45 M, more preferably at most 0.4 M. The control of osmolarity in the method of present invention is essential. Protoplasts are cells deprived of the cell wall, thus very sensitive both to mechanic and osmotic pressure shocks. Usually, osmolarity of the media is governed mainly by mannitol component contribution. To obtain a fast and efficient cellular division, it is essential that the protoplasts are not subjected to harsh conditions, i.e. the mannitol should not exceed 0.5 M, since it will result in non-healthy embryogenic callus protoplasts. The effect of mannitol concentrations on protoplasts obtained by different tissues was screened to empirically set the opportune value of osmolarity for each type of tissue. About 0.4 M mannitol has been found as optimal concentration in the case of grapevine embryogenic callus protoplasts (FIG. 1).

According to another preferred embodiment, the present invention relates to the method, wherein the method further comprises a step f) generation of genome edited whole plants from the mini-callus colonies. Protoplasts cell walls reconstitutes after 2 to 3 days, and further cellular divisions will lead to the regeneration of a gene edited whole plant. As every plant will be regenerated from a single, edited cell, this methodology guarantees genetic homogeneity. Plants obtained by the method of present invention and being regenerated upon successful CRISPR-Cas gene-targeted protoplast transfection, will be 100% edited, thus excluding the risk of chimerism.

According to yet another preferred embodiment, the present invention relates to the method, wherein step f comprises culturing the mini-callus colonies in culture medium comprised of auxins, cytokinins, and optionally about 1 g/L activated charcoal, for at least 3 weeks, preferably at least 4 weeks, most preferably at least 6 weeks.

According to a preferred embodiment, the present invention relates to the method, wherein the ratio auxins:cytokinins in the culture medium is at least about 1:1, preferably about 2:1, more preferably about 3:1. The composition of the liquid culture medium basically consists in a N,N medium enriched with charcoal. The concentration and ratio in auxins and cytokinins was modified to obtain the most optimal ratio to promote both cell division and cell differentiation and provide for successful regeneration of genome edited whole plants from protoplast by the method of present invention. Outside the indicated ratio, there is just proliferation of callus and no embryogenesis. Preferably, kinetin is present in the liquid culture medium, which is a cytokinin that promotes cell division. The culture medium was changed weekly to ensure optimal intake of nutrients and avoid, at the same time, the accumulation of toxic molecules. After 6 weeks, the cultures were transferred on a GS1CA solid medium enriched with antioxidants.

According to a preferred embodiment, the present invention relates to the method, wherein the culture medium comprises between 1.8 to 2.6 μM 6-Benzyladenine (6-BAP), preferably 2 to 2.4 μM, more preferably 2.1 to 2.3 μM, and between 2.5 to 11.5 μM 1-Naphthaleneacetic acid (NAA), preferably 3.8 to 8.1 μM, more preferably 5 to 7.5 μM and optionally between 0.7 to 1.2 μM kinetin, preferably 0.8 to 1 μM, more preferably 0.85 to 0.95 μM kinetin. Experiments have shown that the addition of kinetin in the culture medium for grapevine tissue is preferable.

According to a preferred embodiment, the present invention relates to the method, wherein genome editing is done by CRISPR-Cas technology, preferably CRISPR-Cas9. For example genome editing can be performed by RNPs and single gRNA or by means of transient expression of a vector encoding the nuclease (i.e. Cas9) and the sgRNA.

According to a preferred embodiment, the present invention relates to the method, wherein the whole plant is a genome edited grapevine plant selected from the group consisting of the cultivars Chardonnay, Crimson S., Thompson S., Merlot, Glera, Malbec, and Sugraone, preferably Crimson S or Malbec.

According to yet another preferred embodiment, the present invention relates to the method, wherein the delivery of biological material into the protoplasts is achieved by means of liposome- or polyethylene glycol-(PEG) infiltration, electroporation or lipofection. Successful delivery of biological material into protoplasts that were isolated according to the present method was achieved by means of PEG infiltration. Plasmid DNA 35S-YFP was delivered into protoplasts and these cells overexpressed YFP detectable by confocal fluorescence microscopy after 24 hours upon transfection (FIG. 2). Furthermore, the method of present invention can be applied to obtain disease (for example Downy Mildew (DM)) resistant plants, for example grapevine clones of commercial grape cultivars, for which DM represents a major disease. For example a CRISPR-Cas components comprising sgRNAs for 2-oxoglutarate (2OG) and Fe(II)-dependent oxygenase superfamily protein such as DMR6 (a susceptibility gene for Downy Mildew (DM), for example gene ID: VIT_213s0047g00210) or seven transmembrane MLO family protein such as MLO6 (gene ID: 100233059) susceptibility gene targets can be delivered into protoplasts. As examples show, the method of present invention provided plants of Crimson seedless grape variety genetically edited by CRISPR-Cas on a DMR6 gene, providing a DM resistant grape plant.

According to another preferred embodiment, the present invention relates to the method, wherein the biological material is comprised of one or more selected from the group consisting of CRISPR-Cas components, ribonucleic protein (RNP), single guide RNA (sgRNA), a vector encoding a Cas nuclease and the sgRNA. Preferably the Cas nuclease is Cas9, Cas12a, Cpf1, or Cms1, or the like.

According to a preferred embodiment, the present invention relates to the method, wherein the genome edited protoplast is comprised of one or more mutated sequences selected from the group consisting of SEQ ID No.1, SEQ ID No.2, SEQ ID No. 3, and SEQ ID No. 4.

According to another preferred embodiment, the present invention relates to the method, wherein after the genome editing of the isolated protoplast by delivery of biological material into the protoplast, the protoplasts are kept at 24-26° C. for 16 to 60 hours, preferably 20 to 48 hours, more preferably for 24 to 36 hours, before proceeding to step e). Results have shown that the genome editing proteins need to be able to reach the nucleus, scan the genome to determine the position that needs to be edited and subsequently to enable gene specific genome editing. The process of genome editing of the protoplast genome, and incubation with the genomic editing effector proteins requires time and preferably at least 16 to 60 hours. The most optimal genome editing results, providing the most specific result and highest efficiency, seem to be between 24 to 36 hours. Above 60 hours of incubation without protoplasts embedding is it very likely that the cell culture quality decreases, and as a consequence also the overall editing efficiency.

The present invention, according to a second aspect, relates to a genome edited plant obtainable by a method for the generation of genetically edited protoplasts from clonally propagated plant tissue as disclosed above, wherein said plant is a clonally propagated plant. The method of present invention is suitable for providing mutants in potentially any gene and in any plant for different purposes, more specifically for producing non-chimeric gene edited plants for clonally propagated species such as grape (Vitis vinifera) and potato (Solanum tuberosum). In addition, the potential application of the methodology goes beyond the mere gene knockout to produce resistant grapevines and potatoes for susceptibility genes, as it can be extended to either base- or prime-editing.

According to another preferred embodiment, the present invention relates to the genome edited plant, wherein said plant is a grapevine, apple, cherry, yam, cassava, sweet potato, taro, or potato plant, preferably a grapevine plant or a potato plant.

According to a preferred embodiment, the present invention relates to the genome edited plant, wherein the genome edited plant is comprised of one or more mutated sequences selected from the group consisting of SEQ ID No.1, SEQ ID No.2 and SEQ ID No.3.

According to yet another preferred embodiment, the present invention relates to the genome edited plant, wherein the grapevine plant is one or more selected from the group consisting of Chardonnay, Crimson S., Thompson S., Merlot, Glera, Malbec, and Sugraone, preferably Crimson S or Malbec. According to the method of present invention regeneration of whole genetically modified plants from protoplasts obtained from embryogenic calli of various grape cultivars (Chardonnay, Glera, Malbec, Sugraone) edited through CRISPR-Cas (targeting for example MLO6 and DMR6 genes) can be achieved.

### BRIEF DESCRIPTION OF THE DRAWINGS

The present invention will be further detailed in the following examples and figures wherein:

FIG. 1: shows the positive response of healthy protoplast cells by confocal microscopy (λₑₓ 488 nm), after a viability assay with fluorescein diacetate (FDA viability assay) on protoplasts obtained from Vitis vinifera Crimson s. L. embryogenic callus upon optimized enzymatic digestion according to the method of present invention. De-esterification of FDA (not fluorescent) to fluorescein (fluorescent) occurs by means of the esterases contained in cells (upper photo); fluorescein only accumulates in viable cells. On the bottom photo, a magnification on the structure of a protoplast is shown, in which fluorescent cytosol and the volume corresponding to vacuole can be easily distinguished.

FIG. 2: shows the regeneration from protoplasts obtained from Vv Crimson s. L. embryogenic callus. After 3-4 months from protoplast preparation according to the method of present invention, the first embryos developed in plantlets incubated on GS1CA solid culture medium and transferred into N,N solid medium (upper photo). Subsequently, whole plants developed within additional 3-4 weeks (lower photo).

FIG. 3: shows protoplasts obtained from Vv Crimson s. L. embryogenic callus upon PEG infiltration of 35S YFP plasmid DNA, observed by confocal microscopy (λₑₓ 488 nm). After 24 hours, overexpression of YFP can be detected inside protoplasts, suggesting both their viability and the successful cellular delivery of the plasmid DNA.

FIG. 4: shows plants regenerated from Vv Crimson s. L. embryogenic callus protoplasts transfected with DMR6-targeted CRISPR-Cas components according to the method of present invention. Mutations in the DMR6 gene were confirmed by sequencing. The DMR6 edited plants show normal growth and development in comparison to wild type, non-edited control plants.

FIG. 5: Shows the regeneration from protoplasts obtained from potato leaf material that were successfully transfected with CRISPR-Cas9 having DMR6-1 as gene target. The upper photo shows low melting agarose embedding of protoplasts after transfer to 9 cm petri dishes. The lower photo shows the growth of first developing shoots from callus, observed after 3-4 weeks upon transfer to solid medium. Successful plant development was obtained within 2 months from the start of the experiment.

### DESCRIPTION OF THE INVENTION

### Examples

### Example 1—Isolation of Protoplasts from Grapevine Tissues

To isolate protoplasts from grapevine leaf tissue, about 20 leaves were taken from 25-days-old plants and these leaves were cut into small pieces and subsequently pre-infiltrated with Gamborg B5 medium (including vitamins and 0.3-0.5 M mannitol) in a 15 ml Falcon tube under vacuum for one minute under sterile conditions. Subsequently the liquid medium was discarded and replaced with 12 ml of enzymatic mixture composed by 1% (w/v) cellulase Onozuka R-10, 0.2% (w/v) hemicellulase Sigma, 0.3% (w/v) macerozyme R-10 dissolved in Gamborg B5 (including vitamins and 0.3-0.5 M mannitol) and infiltrated under vacuum for one minute under sterile conditions. Next, the suspension is put into a 90 mm Petri-dish and left on a shaker under gentle tilting for 16 hours, in darkness at 25° C.

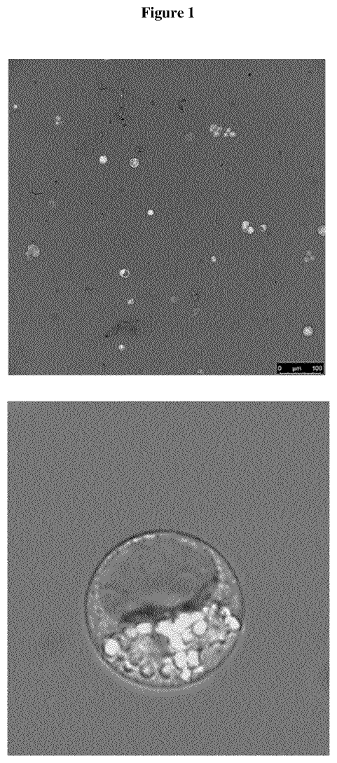

After incubation, the protoplast suspension was filtered through a 60 μm nylon sieve and protoplasts were collected by pelleting via centrifugation at 80 g for 4 minutes, without brake. Next, the collected protoplasts are washed twice in an osmotically adjusted washing solution containing 0.4 M mannitol, 15 mM MgCl₂, 4 mM MES buffer (pH 5.7) (hereafter MMG). Protoplasts were further purified layering 2 ml of protoplasts suspension on 8 ml of a 15-20% w/v sucrose aqueous solution. Healthy protoplasts were collected at the interphase upon centrifugation (90 g, 4 minutes, no brake). The above method successfully provided healthy protoplasts, when applied to either embryogenic or non-embryogenic callus, starting from digestion of 1 g of callus portion (FIG. 1).

### Example 2—Stimulation of the Formation of Micro Callus and Whole Plant Regeneration from Wild-Type Embryogenic Callus-Derived Protoplasts

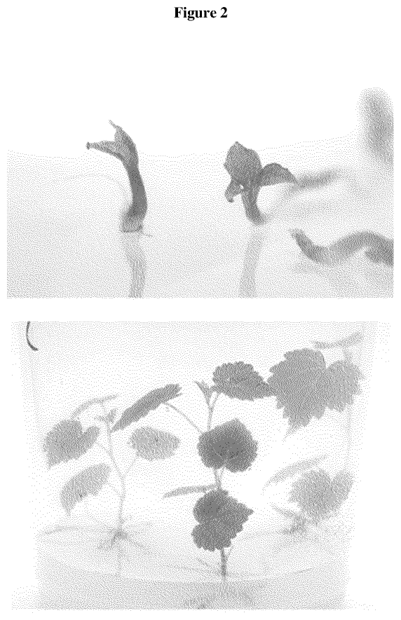

After isolation from embryogenic callus, as reported in Example 1, the protoplasts were cultured and stimulated for the formation of mini-calli. Therefore, the protoplast concentration was adjusted to 200k cells/ml by diluting with MMG, using a cell counting chamber, obtaining the protoplast culture. Subsequently, an isovolume of 1.6% w/v Na-alginate (Sigma) solution (0.4 M mannitol, 5 mM MES buffer, pH 5.7) is added to the protoplast culture and homogenized by gentle pipetting. This mixture was casted (1 ml) on a solid Agar-Ca²⁺ matrix (1.4% w/v Agar, 50 mM CaCl₂, 0.4 M mannitol, pH 5.7) deposited in a 50 mm Petri dish, and left 40 minutes for full solidification. The disks obtained are then transferred into a 50 mm Petri-dish filled with 5 ml of a N,N-based cultivation medium (Nitsch and Nitsch, Science 1969; Vol. 163, p85-87) optimized for regeneration (N,N medium including vitamins, 88 mM sucrose, 300 mM glucose, 1 g/l charcoal, 0.93 μM kinetin, 2.22 μM 6-BAP (Sigma), and 10.7 μM NAA (Sigma). Cultivations were stored at 24 to 26° C. in permanent darkness and the liquid culture medium was substituted weekly. After 2 weeks, the glucose concentration was progressively diminished by 25% per week and after 4 further weeks, no glucose is present in the regenerative culture medium and the disks are transferred on solid GS1CA (Franks et al., 1998, Molecular Breeding 4, p321-333) culture medium, enriched with 300 μM glutathione. After 3 to 4 weeks upon transfer to solid medium, the growth of the first embryos was observed. These embryos were then transferred on N,N solid medium and stored at 16/8 light/dark photoperiod (80-100 μmol m⁻² s⁻¹), at 24 to 26° C. Successful plant development was obtained within 3 to 4 months from the start of the experiment (FIG. 2).

### Example 3—DNA Transfection of Embryogenic Callus-Derived Protoplasts

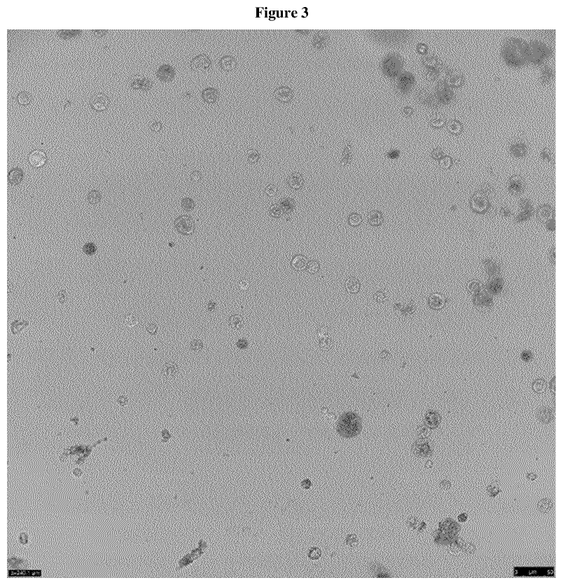

Successful delivery of biological material inside viable protoplasts was achieved by means of PEG infiltration. As a first attempt, plasmid DNA 35S::YFP was delivered into protoplasts and cells overexpressed YFP detectable by confocal fluorescence microscopy after 24 hours upon transfection (FIG. 3).

Briefly, protoplasts were obtained from embryogenic callus enzymatic digestion as described in Example 1. For the transfection, the protoplasts concentration of the suspension is adjusted to 1×10⁶ cells/ml by diluting with MMG and using a cell counting chamber. Once the concentration is reached, 200-250k cells are used for each PEG-mediated transfection and the 35S::YFP plasmid (10-30 μg) is added to the suspension. Next, an isovolume of PEG-Ca solution was added to the mixture and incubated for 10 minutes after homogenizing by gentle pipetting. After the incubation, PEG is removed by centrifuging the mixture upon addition of WI (≈10 ml, 20 mM KCl, 0.5 M mannitol, 4 mM MES buffer, pH 5.7) in a centrifuge tube. The procedure is repeated 2 times. After PEG removal, cells are embedded in alginate disks as described in Example 2. After ≈24 h, a portion of the disk was cut and observed through a fluorescence confocal microscope to detect intracellular YFP (λ_{exc} 488 nm).

### Example 4—Transfection with RNPs of Embryogenic Callus-Derived Protoplasts of Malbec and Crimson S., and Subsequent Sequence Analysis

Next to YFP of example 3, protoplasts were successfully transfected with CRISPR-Cas Ribonucleoproteins (RNPs) having DMR6 as gene target. Protoplasts from Crimson Seedless (Crimson S.) and Malbec embryogenic callus were isolated as reported in Example 1. To transfect with RNPs, the concentration of protoplasts in the suspension was adjusted to 1×10⁶ cells/ml by diluting with MMG and checking by means of cell counting chamber. Once the concentration is reached, 250k cells are used for each PEG-mediated transfection. Cas9 protein (Thermofisher) was mixed with a DMR6-targeting sgRNA (customized, Sigma Aldrich) in NEBuffer 3, pH 7.9 (New England Biolabs) in a 13 ml centrifuge tube. To this mixture, 250 μl of protoplasts suspension was subsequently added (final concentration of 0.5 μM Cas9 and 1.1 μM sgRNA) and homogenized by gentle pipetting. Next, an equal volume of PEG-Ca solution was added to the mixture, which is homogenized by gentle pipetting and incubated for 10 minutes. After the incubation, PEG is removed by centrifuging the mixture upon addition of WI (≈10 ml, 20 mM KCl, 0.5 M mannitol, 4 mM MES buffer, pH 5.7) in a centrifuge tube. The procedure is repeated 2 times.

After PEG removal, cells are resuspended in a 50 mm Petri-dish filled with 5 ml of a N,N-based cultivation medium (N,N medium including vitamins, 88 mM sucrose, 0.3 M glucose, 1 g/l charcoal). Cultivations were stored at 24-26° C. in permanent darkness and the cells were collected by centrifugation after 24 hours. A negative control was prepared without any transfection for each variety.

The genomic DNA of each sample was extracted and deep sequencing of DMR6 gene was performed to check for the presence of editing events. Editing of the DMR6 gene was confirmed by Illumina sequencing (Table 1.), which highlighted the following mutations in both cultivars (Malbec and Crimson S.): the deletion of 1 bp or 2 bp, the insertion of 1 bp (according SEQ ID 1, SEQ ID 2, and SEQ ID3, respectively).

| | | WT | −1 bp | −2 | +1 bp |
|---|---|---|---|---|---|
| FASTQ | SAMPLE | reads | reads | bpreads | reads |
| R1 | Malbec control | 120972 | — | — | — |
| R2 | Malbec control | 117031 | — | — | — |
| R1 | Malbec transformed | 108901 | 214 | — | — |
| R2 | Malbec transformed | 104720 | 203 | — | — |
| R1 | Crimson S. control | 50439 | — | — | — |
| R2 | Crimson S. control | 44471 | — | — | — |
| R1 | Crimson S. transformed | 46766 | 3750 | 265 | 2532 |
| R2 | Crimson S. transformed | 40463 | 3144 | 213 | 2176 |

Table 1. Editing of the DMR6 gene confirmed by sequencing. Number of unedited (wt) and edited reads in DMR6 gene for each transient transformation with RNPs after 24 h, as inferred by sequencing. Samples have been sequenced from both ends (as indicated by R1 and R2), also known as paired end sequencing. Clear editing events in the Crimson S. and Malbec transformed cells are observed. Control (non-transformed) Malbec and Crimson seedless do not show any editing events.

### Example 5—Whole Plant Regeneration from Genome Edited Protoplast

Next to YFP of example 3 and the transient RNP-transformation of example 4, protoplasts were successfully transfected with CRISPR-Cas having DMR6 as gene target and subsequently whole plants were regenerated. As a result, the DMR6 gene was mutated by means of a 1 bp insertion, a 1 bp deletion, or a 2 bp deletion. Protoplasts from embryogenic callus were isolated as reported in Example 1 and transformed as reported in Example 4.

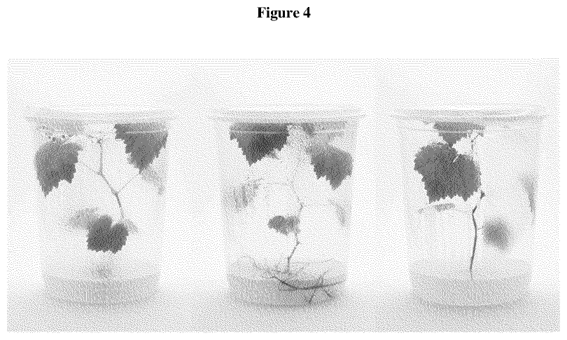

In this case, after PEG removal, cells are embedded in alginate disks as described in Example 2. The disks obtained are transferred into a 50 mm Petri-dish filled with 5 ml of a N,N-based cultivation medium optimized for regeneration (N,N medium including vitamins, 88 mM sucrose, 0.3 M glucose, 1 g/l charcoal, 0.93 μM kinetin, 2.22 μM 6-BAP (Sigma), 10.7 μM NAA (Sigma)). Cultivations were stored at 24-26° C. in permanent darkness and the liquid culture medium was substituted weekly. After 2 weeks, the glucose concentration was progressively diminished by 25% per week. After 4 further weeks, no glucose is present in the regenerative culture medium and the disks are transferred on solid GS1CA culture medium, enriched with 300 μM glutathione. After 3-4 weeks upon transfer to solid medium, the growth of the first embryos was observed. These embryos were then transferred on N,N solid medium and stored at 16/8 light/dark photoperiod (80-100 μmol m⁻² s⁻¹), 24-26° C. Successful plant development was obtained within 3-4 months from the start of the experiment (FIG. 4). Editing of the DMR6 gene was confirmed by Sanger sequencing, in which 7 plants over 8 tested showed homozygous editing. In particular, the observed mutations were a homozygous deletion of 1 bp or 2 bp, as well as a homozygous insertion of 1 bp (SEQ ID No. 1, 2 or 3, respectively).

### Example 6—Isolation of Protoplasts from Potato Tissues

To isolate protoplasts from potato leaf tissue (Solanum tuberosum), about 20 leaves were taken from 4-6 week old potato plants and these leaves were cut into 1 mm pieces in medium containing 0.3 M Sorbitol, 50 mM calcium chloride diyhdrate, 3 mM MES, pH 5.8. Subsequently, 25 ml per gram plant material of enzymatic mixture composed by 0.5% (w/v) cellulase Onozuka R-10, 0.2% (w/v) hemicellulase Sigma, 0.1% (w/v) macerozyme R-10 dissolved in medium including vitamins and 0.3-0.5 M mannitol was put into a 90 mm Petri-dish and left on a shaker under gentle tilting for 16 hours, in darkness at 23-24° C.

After incubation, the protoplast suspension was filtered through a 100 μm nylon sieve and protoplasts were collected by pelleting via centrifugation at 80 g for 10 minutes at room temperature, without brake. The protoplasts are resuspended in a solution containing 0.6 M sucrose and were pelleted via centrifugation at 80 g for 10 minutes. Healthy protoplasts were collected in the floating layer by carefully pipetting. Next, the collected protoplasts are washed twice in an osmotically adjusted washing solution containing 1 g/l Glucose, 18 g/l CaCl₂, 9 g/l NaCl₂ and buffered to pH 5.8 (W5 buffer hereafter) (80 g, 10 minutes, no brake). The above method successfully provided healthy protoplasts.

### Example 7—Whole Plant Regeneration from Genome Edited Protoplast

Protoplasts were successfully transfected with CRISPR-Cas9 having DMR6-1 as gene target. Protoplasts from leaf material were isolated as reported in Example 6. To transfect with RNPs, the concentration of protoplasts in the suspension was adjusted to 1×10⁶ cells/ml by diluting with MaMg and checking by means of a Fuchs Rosenthal cell counter. Once the concentration is reached, 500K cells were used for each PEG-mediated transfection. Cas9 protein (Integrated DNA Technologies; IDT) was mixed with a DMR6-1-targeting sgRNA (customized, IDT) in TE buffer, pH 8 in a 1.5 ml centrifuge tube. To this mixture, 500 μl of protoplasts suspension was subsequently added (final concentration of 7.5 μg Cas9 and 7.5 μg sgRNA) and homogenized by gentle pipetting following incubation of 20 minutes at room temperature.

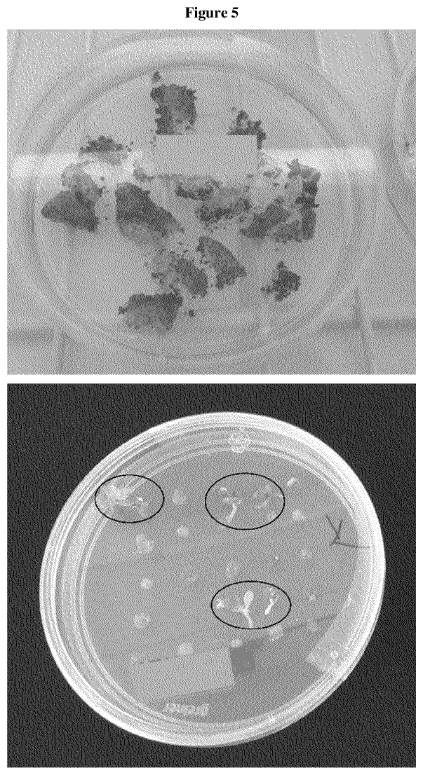

Next, 500 ul of buffer W5 was added to the mixture, which is homogenized by gentle pipetting and incubated for 5 minutes. After the incubation, PEG is removed by centrifuging the mixture upon addition of W5 (≈5 ml) in a 10 ml centrifugation tube (80 g, 10 minutes, no brake). The pellet is resuspended and is transferred into a 3 cm Cellstar petri dish filled with 3 ml of a cultivation medium optimized for regeneration (N,N medium including vitamins, 2 μM 6-BAP (Sigma), 5 μM NAA (Sigma). Cultivations were stored at 24-26° C. in permanent darkness. After 1 day, 4 ml of low melting agarose (LMA) was added to the petri dish and let solidified. The LMA was transferred to a 9 cm petri dish and cut into several pieces (FIG. 5, upper photo). The plates are sealed with parafilm and kept in dark at 25° C. Every week the medium was refreshed, thereby reducing every time the osmolarity of the solution.

After 3-4 weeks upon transfer to solid medium, the growth of the first shoots were observed (FIG. 5, lower photo). These shoots were then transferred on solid medium and stored at 16/8 light/dark photoperiod (80-100 μmol m⁻² s⁻¹), 24-26° C. Successful plant development was obtained within 2 months from the start of the experiment. Editing of the DMR6-1 gene in the regenerated plants was confirmed by Illumina sequencing, in which 4 plants tested showed heterozygous editing. In particular, the observed mutation in this plant was a deletion of 1 bp (SEQ ID No.4).

1. A method for the generation of genome edited protoplasts from clonally propagated plant tissue for generation of whole plants, wherein the method comprises the steps of:
a) providing a cell suspension from clonally propagated plant tissue selected from the group consisting of embryogenic callus, non-embryogenic callus, and/or leaf cells;
b) contacting the cell suspension with an enzyme composition for digestion of a plant cell wall;
c) isolating a protoplast from the cell suspension and washing of the isolated protoplast with a wash solution;
d) genome editing of the isolated protoplast by delivery of biological material into the isolated protoplast; and
e) generating mini-callus colonies from the genome edited protoplast.

2. The method according to claim 1, wherein the clonally propagated plant tissue is selected from the group consisting of grapevine (Vitis vinifera), potato (Solanum tuberosum), apple, cherry, yam, cassava, sweet potato, taro, and woody plant tissues.

3. The method according to claim 1, wherein the enzyme composition is comprised of 0.5 to 3% (w/v) cellulase, 0.1 to 0.6% (w/v) hemicellulase, and 0.1 to 0.5% (w/v) macerozyme R-10.

4. The method according to claim 1, wherein the wash solution is osmotically adjusted to substantially correspond to osmotic values of the isolated protoplast, and wherein the concentration of mannitol is at most 0.5 M.

5. The method according to claim 1, wherein the method further comprises step f) generating whole genome edited plants from the mini-callus colonies by culturing the mini-callus colonies.

6. The method according to claim 5, wherein step f) comprises culturing the mini-callus colonies in culture medium comprised of auxins, cytokinins, and optionally about 1 g/L activated charcoal, for at least 3 weeks.

7. The method according to claim 6, wherein the ratio auxins:cytokinins in the culture medium is at least about 1:1.

8. The method according to claim 6, wherein the culture medium comprises between 1.8 to 2.6 μM 6-Benzyladenine (6-BAP), and between 2.5 to 11.5 μM 1-Naphthaleneacetic acid (NAA), and optionally between 0.7 to 1.2 μM kinetmi.

9. The method according to claim 1, wherein genome editing is done by CRISPR-Cas technology.

10. The method according to claim 1, wherein the whole plant is a genome edited grapevine plant selected from the group consisting of the cultivars Chardonnay, Crimson S., Thompson S., Merlot, Glera, Malbec, and Sugraone.

11. The method according to claim 1, wherein the delivery of biological material into the protoplasts is achieved by means of liposome- or polyethylene glycol-(PEG) infiltration, electroporation, or lipofection.

12. The method according to claim 1, wherein the biological material is comprised of one or more selected from the group consisting of CRISPR-Cas components, ribonucleic protein (RNP), single guide RNA (sgRNA), a vector encoding a Cas nuclease, and the sgRNA.

13. The method according to claim 1, wherein the genome edited protoplast is comprised of one or more mutated sequences selected from the group consisting of SEQ ID No.1, SEQ ID No.2, SEQ ID No. 3, and SEQ ID No. 4.

14. The method according to claim 1, wherein after the genome editing of the isolated protoplast by delivery of biological material into the protoplast, the protoplasts are kept at 24-26° C. for 16 to 60 hours before proceeding to step e).

15. A genome edited plant obtainable by a method for the generation of genetically edited protoplasts from clonally propagated plant tissue according to claim 1, wherein said plant is a clonally propagated plant.

16. The genome edited plant according to claim 15, wherein said plant is a grapevine, potato, apple, cherry, yam, cassava, sweet potato, or taro plant.

17. The genome edited plant according to claim 15, wherein the genome edited plant is comprised of one or more mutated sequences selected from the group consisting of SEQ ID No.1, SEQ ID No.2, SEQ ID No. 3, and SEQ ID No. 4.

18. The genome edited plant according to claim 15, wherein the grapevine plant is one or more selected from the group consisting of the cultivars Chardonnay, Crimson S., Thompson S., Merlot, Glera, Malbec, and Sugraone.
